(19) United States
(12) Patent Application Publication
(10) Pub. No.: US 2020/0402668 A1
(43) Pub. Date: Dec. 24, 2020

(54) METHOD AND KIT FOR DIAGNOSING NON CELIAC GLUTEN SENSITIVITY

(71) Applicants: ALMA MATER STUDIORUM - UNIVERSITÀ Dl BOLOGNA, Bologna (IT); AZIENDA OSPEDALIERO-UNIVERSITARIA DI BOLOGNA POLICLINICO S. ORSOLA - MALPIGHI, Bologna (IT)

(72) Inventors: Giovanni BARBARA, Bologna (IT); Maria Raffaella BARBARO, Bologna (IT); Vincenzo STANGHELLINI, Bologna (IT); Antonio Maria MORSELLI-LABATE, Bologna (IT); Marianna MASTROROBERTO, Bologna (IT); Umberto VOLTA, Bologna (IT); Cesare CREMON, Bologna (IT)

(73) Assignees: ALMA MATER STUDIORUM - UNIVERSITÀ Dl BOLOGNA, Bologna (IT); AZIENDA OSPEDALIERO-UNIVERSITARIA DI BOLOGNA POLICLINICO S. ORSOLA - MALPIGHI, Bologna (IT)

(21) Appl. No.: 16/770,408

(22) PCT Filed: Dec. 3, 2018
(86) PCT No.: PCT/IB2018/059557

§ 371 Date: Jun. 5, 2020

(30) Foreign Application Priority Data
Dec. 6, 2017 (IT) 102017000141093

(51) Int. Cl. G16H 50/30; G16H 10/20; G16H 10/60; G16H 10/40; G16H 50/70; G16H 50/20; G06N 7/00

(52) U.S. Cl. CPC G16H 50/30; G16H 10/20; G16H 10/60; G06N 7/005; G16H 50/70; G16H 50/20; G16H 10/40

(57) ABSTRACT

The present invention provides means for diagnosing non celiac gluten sensitivity.

The present invention provides means for diagnosing non celiac gluten sensitivity.

### PRIOR ART

Non celiac gluten sensitivity (NCGS) is a condition characterised by intestinal and extra-gastrointestinal symptoms, caused by the ingestion of gluten in the absence of a positive diagnosis of celiac disease. The term “non celiac gluten sensitivity” identifies all those cases in which a patient presents symptoms characteristic of celiac disease and benefits from following a gluten-free diet, even though it is possible to rule out the presence of celiac disease or a wheat allergy following medical assessment.

NCGS affects between 0.6 and 6% of the population, however there are currently no biomarkers available for diagnostic purposes. Diagnosis is therefore hypothesised, but is difficult to prove with certainty, on the basis of an improvement of symptoms following the exclusion of gluten from the diet and recurrence of symptoms following re-introduction of gluten into the diet. One of the problems linked with the diagnosis of NCGS lies in the difficulty of distinguishing it, based on symptoms, from irritable bowel syndrome. Instead, it is easier to distinguish NCGS from celiac disease since there are known diagnostic tests for the latter condition. As mentioned above, patients affected by NCGS in fact present symptoms typical of celiac disease even though they are not affected by said disease. Such symptoms include abdominal pain, chronic diarrhoea and/or constipation, stunted growth, anaemia and psychophysical fatigue. Many of these symptoms are also common in patients affected by irritable bowel syndrome (IBS). It should be noted that there is a fair percentage of individuals presenting with IBS for whom the cause of the syndrome could in fact be NCGS.

Zonulin is a human protein homologous of the Vibrio cholera toxin (zonula occludens) which opens the epithelial tight junctions (TJ) reversibly (Wang W, et al. J Cell Sci 2000; 113 Pt 24:4435-40.). Zonulin is expressed in excess in conditions such as celiac disease (CD) and chronic intestinal inflammatory diseases, characterised by TJ dysfunction (Fasano A, et al. Lancet 2000; 355:1518-9). NCGS is defined as a condition characterised by intestinal and extra-intestinal symptoms associated with the ingestion of foods containing gluten in patients in whom CD and wheat allergy have been ruled out (Catassi C, et al. Nutrients 2015; 7:4966-77). The physiopathology of NCGS is still rather unclear, and there are no biomarkers for this pathology. Recently, Holton and collaborators assessed the changes in permeability in explants ex vivo of patients with active celiac disease, NCGS, celiac disease in remission, and non-celiac subjects exposed to gliadin. The results of this study demonstrated a rise in intestinal permeability in NCGS and in active celiac disease without any difference between the two pathologies. The authors of the study concluded that the changes in permeability observed in NCGS, following exposure to gliadin, are equal to those observed in celiac disease (Holton J, et al. Nutrients 2015; 7:1565-76). The molecular mechanisms subjected to such changes in permeability remain unknown to date. Currently, the diagnosis is hypothesised—but difficult to prove with certainty—on the basis of an improvement in symptoms following exclusion of gluten from the diet and recurrence of the symptoms following re-introduction of gluten into the diet. The gold standard is represented by the “double-blind gluten or placebo challenge”, as described previously in the last Consensus Conference of Salerno (Catassi C, et al. Nutrients 2015; 7:4966-77). This practical experiment, however, is not easily reproducible on a large scale due to the need for specialised centres and also the excessive duration of the diagnostic procedure. Currently, there are no biomarkers available for the diagnosis of NCGS. The Consensus Conference of Salerno of 2015 (Nutrients 2015; 7:4966-77) established the current reference standards for the diagnosis of NCGS. These are based on widely subjective elements reported by patients to their doctor and include: intestinal symptoms (abdominal pain and swelling, changes in bowel movements, such as diarrhoea and/or constipation, nausea, aerophagia, gastroesophageal reflux, aphthous stomatitis) and extra-intestinal disturbances (feeling unwell, asthenia, headache, anxiety, brain fog, muscle and joint pain, skin rash, weight loss, anaemia, depression, dermatitis and rhinitis).

The certainty of the diagnosis currently can be obtained exclusively by means of a complex experimental picture which is not easily implemented in clinical practice and which provides a complex clinical evaluation in a number of phases. This evaluation is based on the acknowledgment of a consistent improvement both in symptoms following an exclusion of gluten from the diet and in the effect of gluten ingestion (at least 8 grams/day) verified by the “double-blind gluten or placebo challenge” for a week, followed by a week following a diet devoid of gluten, with successive crossover phase lasting one week. The identification of an objective and not merely subjective diagnostic methodology based solely on the symptoms reported by the patient, as is currently the case, would make it possible to 1) identify the patients affected by NCGS; 2) provide screening for NGCS on an increased proportion of patients presenting to their doctor with symptoms suggestive of celiac disease or irritable bowel syndrome; 3) avoid excessive recourse to healthcare resources; 4) avoid incorrect diagnosis and use of inappropriate diets; 5) legitimize a condition which is currently considered to be of low clinical importance, given the considerable number of patients suffering from it (0.6-6% of the population, estimated in approximately 1,500,000 subjects in Italy).

### SUMMARY OF THE INVENTION

The authors of the present invention have discovered that data relating to the quantification of the concentration of zonulin in the serum of a patient can be combined with clinical data relating to the degree of severity for some symptoms perceived by the same patient so as to obtain a differentiation index which, compared to a defined threshold value, makes it possible provide an objective diagnosis of NCGS in said patient.

In the present description there is thus provided a method for diagnosing non celiac gluten sensitivity (NCGS) in a subject, comprising the following steps:
collecting clinical data indicating a degree of the severity (GS1, GS2) for one or more symptoms (S1, S2) perceived by said subject;
collecting biological data that are indicative of the zonulin concentration (ZL) in a serum sample of the subject being analysed;
elaborating said clinical and biological data to obtain a differentiation index (SC: DAG score);
comparing said differentiation index with a threshold value (BC), the NCGS being diagnosed when said differentiation index (SC) is greater than said threshold value (BC).

A computer program comprising a code for implementing, when running on a computer, a method according to any one of the preceding claims, and a kit comprising the reagents and the material necessary to carry out the above-mentioned method are also provided.

### DETAILED DESCRIPTION OF THE DRAWINGS

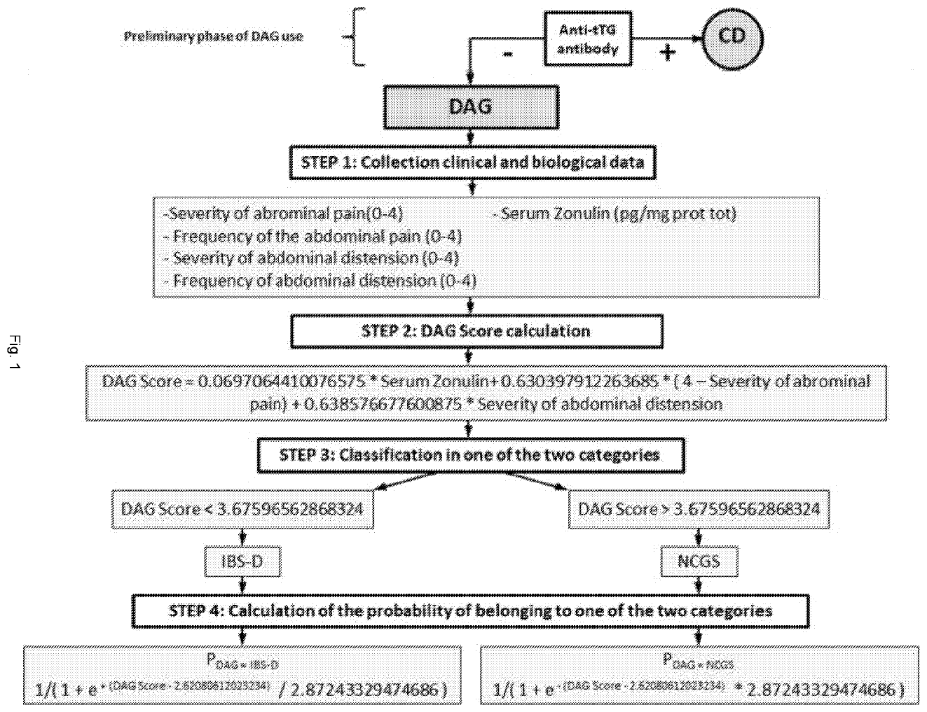

FIG. 1 is a flow chart which shows the method of the invention schematically.

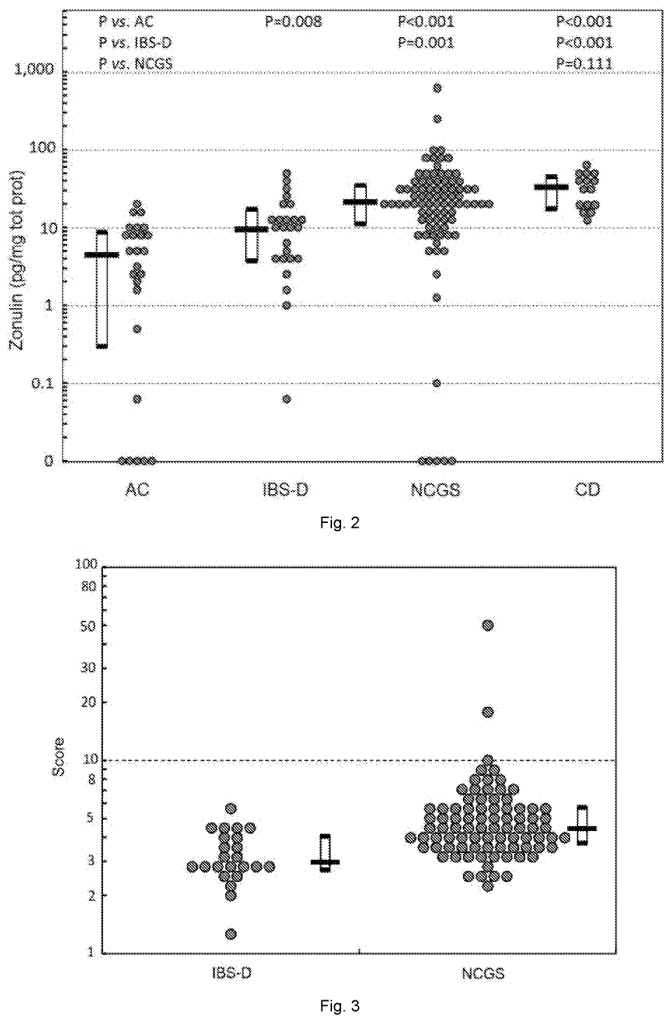

FIG. 2 shows the levels of serum zonulin concentration in the four groups of analysed subjects in the reference sample. The levels of serum zonulin concentration were significantly different in the four groups (P<0.001, Kruskal-Wallis test). The rectangles show the interquartile intervals (that is to say those that comprise half the cases) for each group and the median values are shown inside the rectangles. The probability values (P) relating to the comparisons between the pairs of groups are shown in the upper part of the figure.

FIG. 3 shows the values of the DAG score calculated in the two groups of reference samples (108 patients). The graph also shows the median values and the interquartile intervals (IQI: 25^{th} and 75^{th} percentiles; that is to say the intervals comprising centrally 50% of the samples) of the distributions of the two groups.

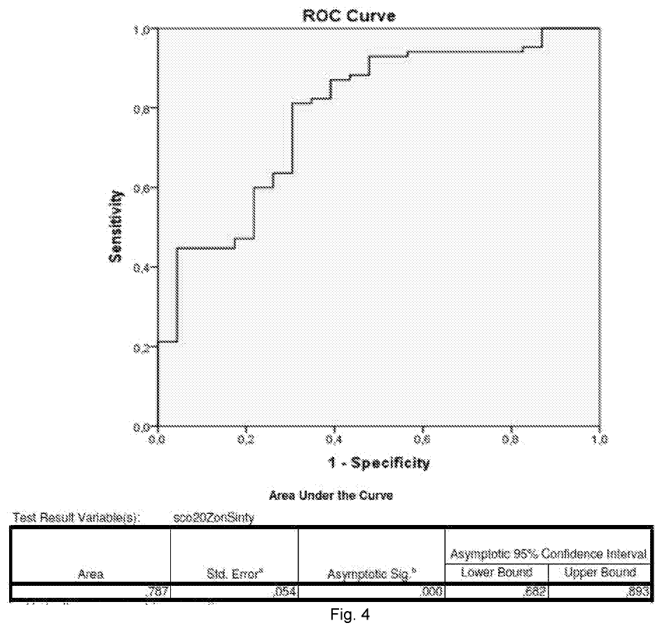

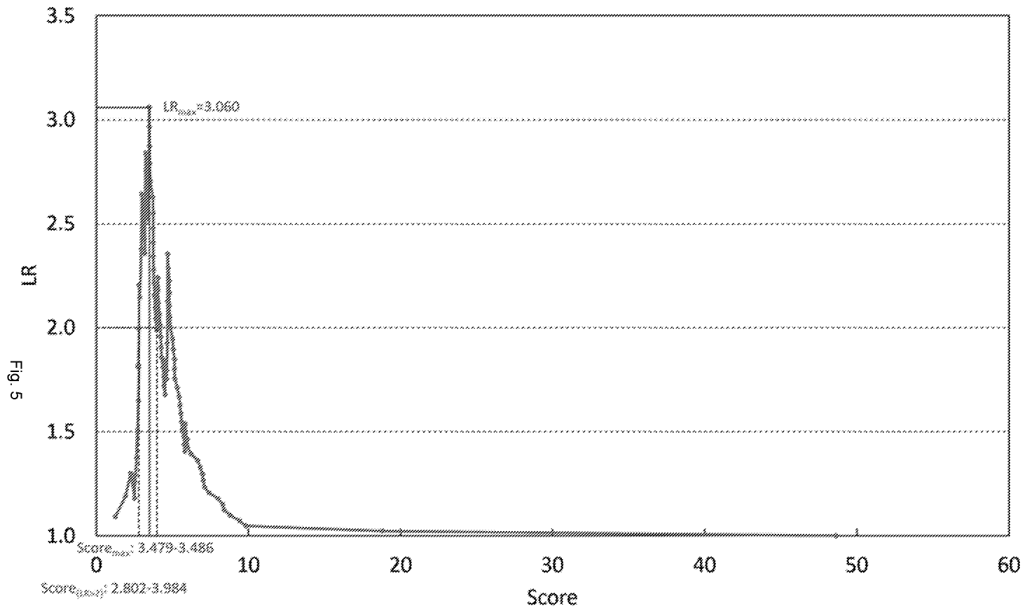

FIG. 4 shows the ROC (receiver operating characteristic) curve for the differentiation of the NCGS patients of the reference sample compared to the IBS-D patients based on the calculated score.

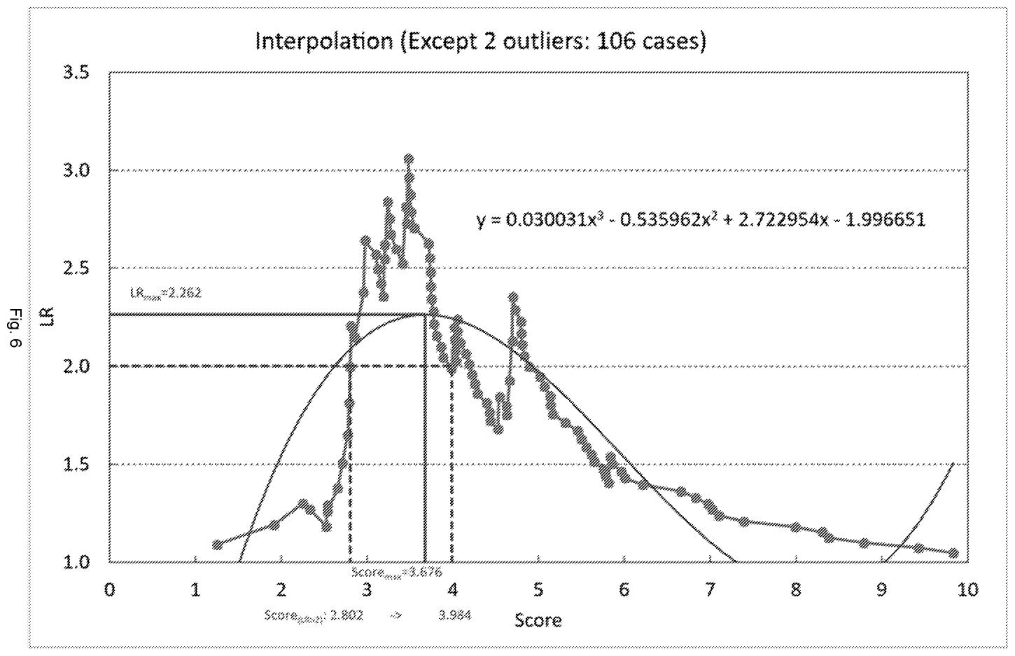

FIG. 5 shows the trend of the LR (likelihood ratio) as a function of the score in the reference sample of 108 patients.

FIG. 6 shows the interpolation of the LR values with a polynomial curve of third degree (cubic) in the interval of the values of the reference sample score which are optimal results (106 patients).

### GLOSSARY

The term “score” as used herein can be synonymous with the term “differentiation index” (SC) as defined in the present description.

Abbreviations:

AUC: area under the curve: celiac disease: ELISA: enzyme-linked immunosorbent assay; SE: standard error; IQI interquartile interval; LR: likelihood ratio; NCGS: non-celiac gluten sensitivity; P: probability; ROC: receiver operating characteristic; TJ: tight junctions; SC: differentiation index; LZ: serum zonulin concentration; S1, S2: symptoms perceptible by the subject under examination; GS1 and GS2: degree of severity of the considered symptoms; C1, C2, C3: weight coefficient.

### DETAILED DESCRIPTION OF THE INVENTION

As indicated above, the present description provides a method for diagnosing non celiac gluten sensitivity in a subject, in which clinical data and biological data are compared, thus providing an objective method with calculable threshold value. This method is based on the discovery of the fact that the combination of certain symptomatic clinical data and biological data relating to the expression of the zonulin protein makes it possible to diagnose, with elevated sensitivity and specificity, a disease not currently able to be diagnosed using objective methods, but able to be diagnosed only subjectively on the basis of information provided by the patient.

With reference to FIG. 1, for the application of the method according to the present invention, a step should firstly be provided in which the serum zonulin concentration LZ in the subject under examination is determined, for example by means of a commercial kit (Zonulin ELISA Kit, Cusabio, Hubei, China). The serum zonulin concentration LZ is expressed as the ratio between the quantity in weight of said zonulin and the quantity in weight of total proteins expressed in the serum of said subject (pg/mg tot prot). The biological data resulting from the test will then be acquired for use in the method of the invention.

Furthermore, according to the invention, clinical data must also be acquired. Clinical data can be collected for example by means of a questionnaire based on the “Bowel Disease Questionnaire”, but modified according to the requirements of the invention.

In particular, the questionnaire must allow the collection of the clinical data indicative of the degree of severity GS1, GS2 of one or more symptoms S1, S2 perceived by the subject under examination.

The symptoms to be considered were identified, from all possible eligible symptoms, as those which presented a role turning out to be statistically significant in addition to the role presented by the other biological data.

This was done by applying a multivariate logistic regression to the pool of considered independent variables (hematic zonulin values, frequency of abdominal pain, severity of abdominal pain, frequency of abdominal distension and severity of abdominal distension), which regression used, as dependent dichotomous variable, the differentiation of the NCGS patients (considered as cases) from IBS-D patients (considered as controls) and followed a backward stepwise procedure. Only the hematic zonulin values, the severity of pain and the severity of abdominal distension remained in the procedure at the end of the logistic regression and thus represented an independent significant contribution.

Thus, the considered symptoms S1 and S2 preferably comprise abdominal pain and abdominal distension.

The questionnaire, visible by way of example hereinafter, can be provided advantageously such that each symptom is classified by the subject by means of a degree of severity in the range 0-4.

The questionnaire recorded personal data and the gender of the patient as well as the date on which the patient filled in the questionnaire.

The symptomatologic questionnaire can be drafted as follows:

| | SEVERITY | | | | | FREQUENCY | | | | |
|---|---|---|---|---|---|---|---|---|---|---|
| SYMPTOMS | S0 | S1 | S2 | S3 | S4 | F0 | F1 | F2 | F3 | F4 |

Abdominal
pain
Abdominal
distension

Severity of the Symptoms

S0=Absent

S1=Slight (not influencing routine activities)

S2=Moderate (noticeable, but does not change routine activities)

S3=Severe (significantly influences

and changes routine activities)

S4=Extremely severe (bed rest)

Frequency of the Symptoms

F0=Absent

F1=Rare (1/week)

F2=Occasional (2-3/week)

F3=Frequent (4-6/week)

F4=Extremely frequent (7/week)

Laboratory Examinations

Zonulin (pg/mg total proteins):

The clinical and biological data thus acquired can be processed to obtain a differentiation index SC which, compared to a threshold value BC, makes it possible to diagnose NCGS when the differentiation index SC is greater than said threshold value BC.

This differentiation index SC represents the degree of differentiation between the probability that each patient may present the characteristics of belonging to the NCGS category or the IBS-D category.

As will become clear, the processing of the acquired clinical and biological data comprises a sum thereof weighted by means of weight coefficients, and therefore by means of a formula of the following type:

SC=C₁·LZ+C₂·(4−GS₁)+C₃·GS₂

in which:
SC represents the differentiation index;
LZ represents the zonulin concentration in serum;
GS₁ and GS₂ represent the degree of severity of the considered symptoms; and
C₁, C₂, C₃ are the weight coefficients.

In particular, GS₁ represents the degree of severity of abdominal pain and GS₂ represents the degree of severity of abdominal distension.

As already mentioned, the differentiation index SC will be compared with a threshold value BC.

The determination of the optimal threshold value BC is clearly a further step that is preliminary to the application of the method according to the invention. In particular, a reference population is firstly identified and is used to acquire the information necessary for the development of the method itself.

By way of example, a population of 108 patients having given their consent (of which 23 are IBS-D and 85 are NCGS) and studied at 3 Italian centres (Department of Medical and Surgical Sciences (DIMEC) of the Alma Mater Studiorum—University of Bologna; First Department of Internal Medicine, S. Matteo Hospital Foundation, University of Pavia; Department of Life, Health and Environmental Sciences, Gastroenterology Unit, University of Aquila) can be considered.

The 23 patients with IBS were diagnosed in accordance with the Roma III criteria [Longstreth G F et al., Gastroenterology 2006; 2006; 130:1480-1491], selecting those with mainly diarrhoeal bowel movements (IBS-D), whilst the 85 patients with NCGS were subjected to the criteria proposed by a panel of experts [Catassi C et al., Nutrients 2015; 7:4966-4977].

The details of the case study are shown in the examples further below and summarised schematically in Table 1 hereinafter.

Table 1: Distribution of clinical and biological data in the sample studied and statistical significance of the difference between the two conditions. The means±SD (standard deviation) and the medians with the interquartile intervals (IQI: 25th and 75th percentile; between parentheses) are shown as descriptive statistics

| | | NCGS | | IBS-D | | P |
|---|---|---|---|---|---|---|
| Serum zonulin (pg/mg | Mean ± SD | 36.1 ± 74.2 | | 11.6 ± 11.2 | | <0.001 |
| tot prot) | Median (IQI) | 21.5 | (11.8-35.5) | 9.4 | (3.8-13.5) | |
| Severity of abdominal | Mean ± SD | 1.64 ± 1.48 | | 1.74 ± 1.01 | | 0.603 (NS) |
| pain (0-4) | Median (IQI) | 2 | (0-3) | 2 | (1-2) | |
| Frequency of | Mean ± SD | 1.80 ± 1.54 | | 1.57 ± 1.08 | | 0.591 (NS) |
| abdominal pain (0-4) | Median (IQI) | 2 | (0-3) | (1 | (1-2) | |
| Severity of abdominal | Mean ± SD | 2.26 ± 1.44 | | 1.70 ± 1.15 | | 0.066 (NS) |
| distension (0-4) | Median (IQI) | 2 | (1-4) | 2 | (1-2) | |
| Frequency of | Mean ± SD | 2.39 ± 1.42 | | 1.87 ± 1.39 | | 0.130 (NS) |
| abdominal distension | Median (IQI) | 3 | (1-3) | 1 | (1-3) | |
| (0-4) | | | | | | |

P: probability of I-type error (Mann-Whitney test);
NS: not significant

For the subjects belonging to the reference population, the clinical and biological data was collected according to the above descriptions, that is to say by means of a compilation of a questionnaire and by means of an analytical determination of the serum zonulin values.

The weight coefficients to be used in the calculation of the differentiation index were created using known instruments, by way of a logistic regression operation. The data relating to the application of the logistic regression is shown in Table 2 below, with the values rounded to 4 decimal places:

TABLE 2
Logistic regression variables

| | | Coefficient | |
|---|---|---|---|
| | Variable | (Estimate ± ES) | Significance |
| Step 1 | Zonulin | 0.0819 ± 0.0273 | P = 0.003 |
| | 4 - pain severity | 2.0530 ± 0.7769 | P = 0.008 |
| | Pain frequency | 1.4265 ± 0.6932 | P = 0.040 |
| | Severity of distension | 1.5219 ± 0.6723 | P = 0.024 |
| | Frequency of distension | −0.9651 ± 0.5877 | P = 0.101 |
| | Constant | −8.2233 ± 3.1515 | P = 0.983 |
| Step 2 | Zonulin | 0.0718 ± 0.0255 | P = 0.005 |
| | 4 - pain severity | 1.4368 ± 0.5925 | P = 0.015 |
| | Pain frequency | 0.9014 ± 0.5583 | P = 0.106 |
| | Severity of distension | 0.5591 ± 0.2811 | P = 0.047 |
| | Constant | −5.8551 ± 2.4248 | P = 0.873 |
| Step 3 | Zonulin | 0.0697 ± 0.0240 | P = 0.004 |
| | 4 - pain severity | 0.6304 ± 0.2904 | P = 0.030 |
| | Severity of distension | 0.6386 ± 0.2810 | P = 0.023 |
| | Constant | −2.6208 ± 1.2358 | P = 0.848 |

SE: standard error

The calculated values of the logistic regression are those obtained at the end of the procedure (that is to say the third run), and the values of the coefficients that can be taken into consideration are those comprised within the limits of the variability intervals indicated by the standard error (SE) around the central estimated value:

| | |
|---|---|
| C₁ | 0.0697 ± 0.0240: from 0.0457 to 0.0937 |
| C₂ | 0.6304 ± 0.2904: from 0.3400 to 0.9208 |
| C₃ | 0.6386 ± 0.2810: from 0.3576 to 0.9196 |
| Constant | −2.7621 ± 1.2358: from −3.9979 to −1.5263 |

Since it is preferable to adopt the central values of the estimations made, the values of the coefficients C₁, C₂ and C₃ with the precision obtained by means of the logistic regression will be used, and the formula resulting therefrom is:

SC=0.0697064410076575·LZ+0.630397912263685·(4−GS₁)+0.638576677600875·GS₂.

It should be noted that since the severity of abdominal pain (parameter coded by values rising from 0 to 4) yielded a negative coefficient (that is to say one which provides a negative contribution to the differentiation index), in order to obtain differentiation index values that are exclusively positive and equal to or greater than 0 in the calculation of the differentiation index, the complement to 4 of the severity of abdominal pain was considered (that is to say values decreasing from 4 to 0 as the severity grows from 0 to 4) and the relative coefficient with positive sign was considered. Thus, without consideration of the constant logistic coefficient (−2.62080612023234) in the calculation of SC, the differentiation index value was equal to the value of the domain z of the function calculated from the logistic regression minus the value of the constant coefficient, that is to say:

SC=z+2.62080612023234.

The differentiation index SC assumes positive values greater than or equal to zero. However, lower SC values represent a greater probability of belonging to the category IBS-D, whereas higher values represent a greater probability of belonging to the category NCGS.

In particular, it is desired to determine an optimal threshold value (best cut-off) for use in order to decide whether a subject can be considered to be belonging to the IBS-D or NCGS category. This threshold value, with which the differentiation index of said subject is compared, can thus be determined from derived data and relative data of the reference population.

The means±SD (standard deviation) of the values of the differentiation index in patients with IBS-D and NCGS were, respectively, equal to 3.32±1.06 and 5.45±5.23, whereas the distribution of the values observed in the reference population groups is shown in FIG. 3.

In accordance with one embodiment, the diagnostic accuracy obtained when using the differentiation index in order to differentiate between patients with NCGS and those with IBS-D is also assessed. This is achieved using an ROC (receiver operating characteristic) curve, which shows a value of the area under the curve (AUC) equal to 0.787 with a variability indicated by a standard error (SE) value equal to 0.054. The diagnostic accuracy of the differentiation index is therefore equal to 78.7% (highly significant value from a statistical viewpoint: P<0.001).

This ROC curve is shown in FIG. 4.

The calculation of the optimal threshold value (best cut-off) must take into consideration the balance between the values for sensitivity (true positives in cases of NCGS) and for specificity (true negatives in the IBS-D controls) of the ROC curve. The quantification of the degree of discrimination (that is to say the balance between values for sensitivity and specificity) was used as verisimilitude index, or ratio of verisimilitude (likelihood ratio; LR) of the ROC curve, and was calculated using the relative frequencies of the cases correctly or incorrectly classified in the two conditions in accordance with the following formula:

LR=(sensitivity+specificity)/((1−sensitivity)+(1−specificity))

The trend of this LR (ordinate) as a function of SC (abscissa) relative to the reference population is shown in FIG. 5.

The maximum value of LR in the reference sample was equal to 3.060, which corresponds to differentiation index values between 3.47937573 and 3.48586218. Considering that LR values greater than 2 are indicative of a good discrimination, it is therefore legitimate to hypothesise a correction functioning for score values between 2.802 and 3.984 (FIG. 5).

Thus, having considered a patient of the reference sample with a differentiation index value equal to or greater than 3.48586218 as NCGS and having instead considered a patient with a differentiation index value equal to or less than 3.47937573 as IBS-D, the ability to distinguish within the reference sample was represented by a sensitivity value equal to 81.2% (correctly classified NCGS cases) and by a specificity value equal to 69.6% (correctly classified IBS-D cases), corresponding to an LR value equal to 3.060.

The LR curve was therefore interpolated, excluding the samples (two in the example) with more elevated SC values insofar as such values are clearly aberrant (scores equal to 18.782 and 48.621; FIG. 5),

The interpolation was preferably performed with a polynomial function.

The low-order polynomial curve which provided a suitable interpolation was the cubic polynomial (third order polynomial; FIG. 6) and is described by the following formula:

y=0.030031x³−0.535962x²+2.722954x−1.996651

The SC value corresponding to the maximum value of the interpolation curve is that which, among the two values that cancelled out the first derivative of the interpolation curve, had a negative second-derivative value.

The values that cancelled out the first derivative were calculated by applying the method for solving the second-degree equations.

Having considered that the first derivative was:

dy/dx=0.090093x²−1.071924x+2.722954

the following differentiation index values were obtained:

x₁,x₂=(1.071924±(1.071924²−4·0.090093·2.722954)^{1/2})/(2·0.090093)

that is to say, respectively:

x₁=8.22200646681808 and x₂=3.67596562868324

Having considered that the second derivative was:

d²y/dx²=0.180186x−1.071924

the second-derivative values corresponding to the identified values were, respectively, equal to:

d²y/dx₁²=0.4095664572300820 and

d²y/dx₂²=−0.4095664572300820

Thus, the optimal threshold value BC (best cut-off) for differentiation between IBS-D and NCGS was the second, that is to say 3.67596562868324.

This value can be used also in approximated form, equal to 3.6760.

This value coincides with a maximum interpolated LR value equal to 2.26223713930298 (FIG. 6).

Consequently, the patients having a differentiation index SC less than 3.67596562868324 are classified as IBS-D, whereas patients having a differentiation index SC greater than 3.67596562868324 are classified as NCGS.

In accordance with some embodiments of the present invention the method can also provide a step making it possible to calculate a probability (P_{NCGS}) associated with the diagnosis of NCGS.

For the calculation of the probability of belonging to the NCGS or IBS-D category, the predictive probability value calculated from the logistic regression was used, that is to say the codomain of the logistic function with domain z:

P_{Pred}=1/(1+e^{-z})

which, taking into consideration the value of the constant coefficient of the logistic regression and considering that z=SC−2.62080612023234, in terms of differentiation index, becomes:

P_{Pred}=1/(1+e^{-(SC-2.62080612023234)})

Having considered that the predictive probability associated with the cut-off value is:

$$\begin{matrix}{P}_{Cut \text{-} off} = {P}_{Pred} ( 3.67596562868324 ) = \\ = 1 / ( 1 + {e}^{- ( 3.67596562868324 - 2.62080612023234 )} ) = \\ = 1 / ( 1 + {e}^{- 1.05515950845090} ) \\ = 0.74176443494674\end{matrix}$$

and that this value reflects the imbalance of the numbers in the two groups in the sample studied (that is to say: 85/108 (78.7%) NCCS vs. 23/108 (21.3%) IBS-D), the probability of belonging to the two categories was calculated by normalising the value of P_{Pred} to an equal probability value for the two groups (that is to say P=0.5) by applying the following formulas:

P_{NCGS}=P_{Pred}*(1−P_{Cut-off})/(P_{Pred}*(1−P_{Cut-off})+(1−P_{Pred})*P_{Cut-off})

P_{IBS-D}=(1−P_{Pred})*P_{Cut-off}/(P_{Pred}*(1−P_{Cut-off})+(1−P_{Pred})*P_{Cut-off})

Having considered the value:

Rp_{Cut-off}=P_{Cut-off}/(1−P_{Cut-off})

it can be demonstrated that (see the annex):

$${P}_{NCGS} = 1 / ( 1 + {e}^{- ( SC - 2.62080612023234 )} * {Rp}_{Cut \text{-} off} )$$

$${P}_{IBS \text{-} D} = 1 / ( 1 + {e}^{+ ( SC - 2.62080612023234 )} / {Rp}_{Cut \text{-} off} )$$

In the reference same the value of Rp_{Cut-off} is equal to 2.87243329474686, that is to say 0.74176443494674/(1−0.74176443494674).

Thus, for patients classified respectively as NCGS and IBS-D, the probability values are:

P_{NCGS}=1/(1+e^{-(SC-2.62080612023234)}*2.87243329474686)

P_{IBS-D}=1/(1+e^{+(SC-2.62080612023234)}/2.87243329474686)

The reliability of the classification of each single case can be determined by subdividing the classification itself into probability bands, for example:
uncertain (probability 50-60%)
quite probable (probability 60-70%)
fairly probable (probability 70-80%)
very probable (probability 80-90%)
highly probable (probability >90%)

The diagnostic accuracy of the proposed method in differentiating the patients with NCGS from those with irritable bowel syndrome (IBS-D) is equal to 78.7% in the reference sample (FIG. 4).

Table 3 below shows some examples of application of the method according to the invention in patients presenting different patterns of clinical and biological data. In particular, patient #0 represents the limit case of a patient with zonulin values of zero, extreme severity of abdominal pain and absence of abdominal distension, that is to say a patient who has a differentiation index value of zero. Patient #1 represents the case of a patient with an intermediate degree of severity and in particular absence of abdominal distension and low zonulin values and is classified by the system correctly as IBS-D with a probability of 92%. Patient #2 presents mild symptoms and low zonulin values. In this case the system classifies the patient correctly as IBS-D with a probability of 53%. Patient #3 presents extremely severe symptoms with intermediate zonulin values. This case is classified correctly as NCGS with a probability of 51%. Patient #4 has severe symptoms and high zonulin levels. The system classifies this patient correctly as a case of NCGS with a probability equal to 96%.

TABLE 3
Examples of five patients whose diagnoses were determined
by means of the method of the invention.

| | Patient #0 | Patient #1 | Patient #2 | Patient #3 | Patient #4 |
|---|---|---|---|---|---|
| Levels of serum zonulin | 0 | 8.935 | 5.521 | 16.685 | 54.358 |
| (pg/mg total proteins) | | | | | |
| Severity of abdominal pain | 4 (extreme | 3 (severe) | 1 (mild) | 4 (extreme | 3 (severe) |
| (0-4) | severity) | | | severity) | |
| Frequency of abdominal | 2 (2-3 | 3 (4-6 | 1 (1 day/ | 4 (daily) | 3 (4-6 |
| pain (0-4) | days/week) | days/week) | week) | | days/week) |
| Severity of abdominal | 0 (absent) | 0 (absent) | 2 (relevant) | 4 (extreme | 4 (extreme |
| distension (0-4) | | | | severity) | severity) |
| Frequency of abdominal | 0 (absent) | 0 (absent) | 2 (2-3 | 4 (daily) | 4 (daily) |
| distension (0-4) | | | days/week) | | |
| SCORE | 0 | 1.253 | 3.553 | 3.717 | 6.974 |
| Diagnosis | IBS-D | IBS-D | IBS-D | NCGS | NCGS |
| P (IBS-D) | 97.5% | 91.9% | 53.1% | 49.0% | 3.6% |
| P (NCGS) | 2.5% | 8.1% | 46.9% | 51.0% | 96.4% |
| Frequency of abdominal | 0 (absent) | 0 (absent) | 2 (relevant) | 4 (extreme | 4 (extreme |
| distension (0-4) | | | | severity) | severity) |

P = probability

A further subject of the invention is a computer program comprising a code for implementing, when running on a computer, the method as described in accordance with any of the described embodiments or in accordance with the examples as reported further below.

Lastly, another subject of the invention is a diagnostic kit for diagnosing non celiac gluten sensitivity (NCGS) in a subject, comprising:

a questionnaire for the acquisition of clinical data indicating a degree of the severity (GS1, GS2) for one or more symptoms (S1, S2) perceived by said subject; and reagents for dosing the amount of serum zonulin (ZL) expressed in a serum sample of said subject.

The questionnaire can be as described previously by way of example in the present description, and the dosing of the amount of serum zonulin expressed in the sample under examination can be performed in accordance with any method known to a person skilled in the art without the need for further teaching to be provided in the present description.

The kit of the invention can also comprise controls calibrated in respect of the amount of zonulin and representative of healthy populations, of patients suffering from celiac disease and/or patients suffering from IBS.

The kit of the invention can also comprise a computer program or a support comprising a computer program, as defined above.
The following examples show possible, non-limiting embodiments of the method of the invention.

### EXAMPLES

1. Procedure for Creating the DAG (Diagnostic Algorithm for Gluten)

The procedure for creating the DAG was developed in the following 9 phases:
identification of the reference sample for the data acquisition
collection of clinical and biological data
   clinical anamnesis by means of the “Bowel Disease Questionnaire” dosing of the levels of serum zonulin
logistic regression
calculation of the score:

Score=0.0697064410076575*serum zonulin+0.630397912263685*(4−severity of abdominal pain)+0.638576677600875*severity of abdominal distension
diagnostic accuracy of the score: 78.7% (ROC curve)
index for quantification of the degree of differentiation (LR)
best cut-off of the score: 3.67596562868324
classification into NCGS and IBS-D
calculation of the probability of the classification

1.1 Identification of the Reference Sample for the Data Acquisition

A reference sample to be used to acquire the information necessary to develop the algorithm was identified. For this purpose, a population of 108 patients (of which 23 were IBS-D and 85 were NCGS) and studied at 3 Italian centres (Department of Medical and Surgical Sciences (DIMEC) of the Alma Mater Studiorum—University of Bologna; First Department of Internal Medicine, S. Matteo Hospital Foundation, University of Pavia; Department of Life, Health and Environmental Sciences, Gastroenterology Unit, University of Aquila) was selected.

The 23 patients with IBS were diagnosed in accordance with the Roma III criteria [Longstreth G F et al., Gastroenterology 2006; 2006; 130:1480-1491], selecting those with mainly diarrhoeal bowel movements (IBS-D), whilst the 85 patients with NCGS were subjected to the criteria proposed by a panel of experts [Catassi C et al., Nutrients 2015; 7:4966-4977]. The details of the case study are provided in the examples below.

1.2. Collection of the Clinical and Biological Data

The clinical and biological data of the reference sample were collected, comprising:
clinical anamnesis by means of the modified “Bowel Disease Questionnaire” (Barbara G et al., Gastroenterology 2004; 128:693-72)
determination of the serum zonulin values by means of a commercial kit (Zonulin ELISA Kit, Cusabio, Hubei, China)

The distribution of the clinical and biological data of the reference sample and the statistical significance between the two conditions are shown in Table 1. A non-parametric method (Mann-Whitney test) was used as statistical test, and the limit adopted universally in clinical practice was used as criterion for determining the statistical significance, that is to say a first-type error probability value in null hypothesis refutal of less than 5% (that is to say P<0.05). Only zonulin showed a highly significant difference between the two groups, whereas the difference of the severity of abdominal distension between the two groups was only close to the significance limit.

1.3 Logistic Regression

Those parameter values displaying an independent role in the differentiation between the two conditions, that is to say a role that is statistically significant in addition to the role of the other parameters already considered in the analysis in successive steps were identified. This was done by applying a multivariate logistic regression to the pool of considered independent variables (hematic zonulin values, frequency of abdominal pain, severity of abdominal pain, frequency of abdominal distension and severity of abdominal distension), which regression used, as dependent dichotomous variable, the differentiation of the NCGS patients (considered as cases) from IBS-D patients (considered as controls) and followed a backward stepwise procedure. Only the hematic zonulin values, the severity of pain and the severity of abdominal distension remained in the procedure at the end of the logistic regression and thus represented an independent significant contribution (Table 2).

1.4 Calculation of the Score

A score that can represent the degree of differentiation between the probability that each patient had of presenting the characteristics to belong to the NCGS category or the IBS-D category was calculated. The score was calculated having considered the coefficients obtained from the logistic regression (see Table 2) according to the following formula:

Score=0.0697064410076575*serum zonulin+0.630397912263685*(4−severity of abdominal pain)+0.638576677600875*severity of abdominal distension

Since the severity of abdominal pain (parameter coded by values rising from 0 to 4) yielded a negative coefficient (that is to say one which provides a negative contribution to the score), in order to obtain score values that were exclusively positive and equal to or greater than 0 in the calculation of the score, the complement to 4 of the severity of abdominal pain was considered (that is to say values decreasing from 4 to 0 as the severity grows from 0 to 4) and the sign of the relative coefficient was reversed. In addition, without consideration of the constant logistic coefficient (−2.62080612023234), the score value was equal to the value of the domain z of the function calculated from the logistic regression minus the value of the constant coefficient, that is to say:

score=z+2.62080612023234

According to these criteria, lower score values represented a greater probability of belonging to the IBS-D category, whereas higher score values represented a greater probability of belonging to the NCGS category.

The means±SD (standard deviation) of the score in patients with IBS-D and NCGS were, respectively, equal to 3.32±1.06 and 5.45±5.23, whereas the distribution of the score values in the two reference sample groups is shown in FIG. 3.

1.5 Assessment of the Diagnostic Accuracy

The diagnostic accuracy of the score in the differentiation of patients with NCGS from those with IBS-D was assessed. This was achieved using an ROC (receiver operating characteristic) curve, which demonstrated a value of the area under the curve (AUC) equal to 0.787 with a standard error (ES) of 0.054. The diagnostic accuracy of the score was therefore equal to 78.7% (highly significant value from a statistical viewpoint: P<0.001) (FIG. 4).

1.6. Index for Quantification of the Degree of Differentiation LR

An index making it possible to identify the score value optimal for the differentiation between the two conditions taking into consideration the balance between the values for sensitivity (true positives in cases of NCGS) and for specificity (true negatives in the IBS-D controls) of the ROC curve was assessed. The ratio of verisimilitude (likelihood ratio; LR) of the ROC curve proposed by Pezzilli and collaborators [Pezzilli et al. Dig Dis Sci. 1995; 40:2341-8 and Lusted L B et al., N Engl J Med 284:416-424, 1971] was used as index and was calculated using the relative frequencies of the cases correctly or incorrectly classified in the two conditions in accordance with the following formula:

LR=(sensitivity+specificity)/((1−sensitivity)+(1−specificity))

The trend of this LR (y) as a function of score (x) relative to the reference population is shown in FIG. 5. The maximum value of LR in the reference sample was equal to 3.060, which corresponds to score values between 3.47937573 and 3.48586218. Thus, having considered a patient of the reference sample with a score value equal to or greater than 3.48586218 as NCGS and having instead considered a patient with a score value equal to or less than 3.47937573 as IBS-D, the ability to distinguish within the reference sample was represented by a sensitivity value equal to 81.2% (correctly classified NCGS cases) and by a specificity value equal to 69.6% (correctly classified IBS-D cases).

1.7 Best Cut-Off of the Score

A value of the score optimal for the differentiation between IBS-D and NCGS (best cut-off) which can be extrapolated to a general population and which is not only specific for the reference sample (therefore is applicable also to populations and samples obtained in other studies and/or centres) was identified. This procedure was developed in the following phases:
a. Identification of the optimal range of the values to be interpolated. By interpolation of the curve of the LR, the two cases with higher score values were excluded insofar as such values are clearly aberrant (18.782 and 48.621; FIG. 5)
b. Interpolation of the curve of the LR in the interval of the optimal score values with a polynomial function. The low-order polynomial function which provided a suitable interpolation was the cubic polynomial (third order polynomial; FIG. 6) and is described by the following formula:

y=0.030031x³−0.535962x²+2.722954x−1.996651
c. Identification of the score value corresponding to the maximum value of the interpolation curve. This value was that which, among the two values that cancelled out the first derivative of the interpolation curve, had a negative second-derivative value.
The values that cancelled out the first derivative were calculated by applying the method for solving the second-degree equations.
Having considered that the first derivative was:

dy/dx=0.090093x²−1.071924x+2.722954
the following score values were obtained:

x₁,x₂=(1.071924±(1.071924²−4*0.090093*2.722954)^{1/2})/(2*0.090093)
that is to say, respectively:

x₁=8.22200646681808 and x₂=3.67596562868324
Having considered that the second derivative was:

d²y/dx²=0.180186x−1.071924
the second-derivative values were, respectively, equal to:

d²y/dx₁²=0.4095664572300820 and d²y/dx₂²−0.4095664572300820
Thus, the optimal scored value (best cut-off) for differentiation between IBS-D and NCGS was the second, that is to say 3.67596562868324. This value coincides with a maximum interpolated LR value equal to 2.26223713930298 (FIG. 6).

1.8 NCGS and IBS-D Classification

Patients were classified into one of the two groups NCGS and IBS-D. According to the adopted criteria, patients with score values lower than the best cut-off were considered IBS-D and patients with score values greater than the best cut-off were considered NCSG.

1.9 Calculation of the Probability of the Classification

For the calculation of the probability of belonging to the NCGS or IBS-D category, the predictive probability value was used, calculated from the logistic regression as codomain of the logistic function with domain z:

P_{Pred}=1/(1+e^{-z})

which, taking into consideration the value of the constant coefficient of the logistic, in terms of score, becomes:

P_{Pred}=1/(1+e^{-(score-2.62080612023234)})

Having considered that the predictive probability associated with the cut-off value is:

$$\begin{matrix}{P}_{Cut \text{-} off} = {P}_{Pred} ( 3.67596562868324 ) \\ = 1 / ( 1 + {e}^{- ( 3.67596562868324 - 2.62080612023234 )} ) = \\ = 1 / ( 1 + {e}^{- 1.05515950845090} ) \\ = 0.74176443494674\end{matrix}$$

and that this value reflects the imbalance of the numbers in the two groups in the sample studied (that is to say: 85/108 (78.7%) NCCS vs. 23/108 (21.3%) IBS-D), the probability of belonging to the two categories was calculated by normalising the value of P_{Pred} to an equal probability value for the two groups (that is to say P=0.5) by applying the following formulas:

P_{NCGS}=P_{Pred}*(1−P_{Cut-off})/(P_{Pred}*(1−P_{Cut-off})+(1−P_{Pred})*P_{Cut-off})

P_{IBS-D}=(1−P_{Pred})*P_{Cut-off}/(P_{Pred}*(1−P_{Cut-off})+(1−P_{Pred})*P_{Cut-off})

Having considered the value:

Rp_{Cut-off}=P_{Cut-off}/(1−P_{Cut-off})

it is easy to demonstrate, with simple algebraic transitions (see the annex), that:

$${P}_{NCGS} = 1 / ( 1 + {e}^{- ( score - 2.62080612023234 )} * {Rp}_{Cut \text{-} off} )$$

$${P}_{IBS \text{-} D} = 1 / ( 1 + {e}^{- ( score - 2.62080612023234 )} * {Rp}_{Cut \text{-} off} )$$

In the reference sample the value of Rp_{Cut-off} was equal to 2.87243329474686, that is to say 0.74176443494674/(1−0.74176443494674).

2. Dosing of Serum Zonulin

The levels of serum zonulin were dosed by means of an immunoenzymatic test (ELISA). The hematic samples were centrifuged at 3000 rpm for 7 minutes and the serum thus obtained was collected, aliquoted and stored at −20° C. until the time of dosing. In order to quantify the serum zonulin levels, a commercially available kit was used (Zonulin ELISA Kit, Cusabio, Hubei, China) in accordance with the manufacturer's instructions. The sensitivity of the kit is 0.156 ng/mL.

Each sample was analysed blind and in duplicate, and the amount of zonulin was normalised by the quantity of total proteins present in the sample. The quantification of the total proteins was performed by means of the use of NanoDrop 2000 spectrophotometer (Thermo Scientific, Milan, Italy); the results are recorded as pg of zonulin/mg of total proteins.

3. Processing of the Data and Statistical Analysis

The data were processed using the IBM SPPS Statistics program (version 23; IBM Co., Armonk, N.Y., USA) using a Surface personal computer (Microsoft Co., Redmond, Wash., USA) with MS Windows 10 Pro operating system (Microsoft Co., Redmond, Wash., USA).

### ANNEX

P_{NCGS} e P_{IBS-D} as a function of P_{Pred}

P_{NCGS}=P_{Pred}*P_{Cut-off})/(P_{Pred}*P_{Cut-off})+(1−P_{Pred})*P_{Cut-off})

P_{NCGS}=1/(1+(1−P_{Pred})*P_{Cut-off})/(P_{Pred}*(1−P_{Cut-off}))

P_{NCGS}=1/(1+(1−P_{Pred})/P_{Pred}*P_{Cut-off}/(1−P_{Cut-off}))

P_{NCGS}=1/(1+(1/P_{Pred}−1)*Rp_{Cut-off})

P_{NCGS}=1/(1+(1/P_{Pred})*Rp_{Cut-off}−Rp_{Cut-off})

P_{NCGS}=1/(1−Rp_{Cut-off}+(1/P_{Pred})*Rp_{Cut-off})

P_{NCGS}=1/(1−Rp_{Cut-off}+Rp_{Cut-off}/P_{Pred})

P_{NCGS}=1/(1+Rp_{Cut-off}(1/P_{Pred}−1)

P_{IBSD}=1−P_{NCGS}

P_{IBSD}=1−(1/(1−Rp_{Cut-off}+Rp_{Cut-off}/P_{Pred}))

P_{IBSD}=(1−Rp_{Cut-off}+Rp_{Cut-off}/P_{Pred}−1)/(1−Rp_{Cut-off}+Rp_{Cut-off}/P_{Pred})

P_{IBSD}=(Rp_{Cut-off}+Rp_{Cut-off}/P_{Pred})/(1−Rp_{Cut-off}+Rp_{Cut-off}/P_{Pred})

P_{IBSD}=(Rp_{Cut-off}/P_{Pred}−Rp_{Cut-off})/(1+Rp_{Cut-off}/P_{Pred}−Rp_{Cut-off})

P_{IBSD}=1/(1/(Rp_{Cut-off}/P_{Pred}−Rp_{Cut-off})+1)

P_{IBSD}=1/(1+1/(Rp_{Cut-off}/P_{Pred}−Rp_{Cut-off}))

P_{IBSD}=1(1+(1/(Rp_{Cut-off}(1/P_{Pred}−1))))

P_{NCGS} and P_{IBS-D} as a function of the DAG score

$${P}_{NCGS} = {P}_{Pred} * ( 1 - {P}_{Cut \text{-} off} ) / ( {P}_{Pred} * ( 1 - {P}_{Cut \text{-} off} ) + ( 1 - {P}_{Pred} ) * {P}_{Cut \text{-} off} )$$

$${P}_{NCGS} = {P}_{Pred} / ( {P}_{Pred} + ( 1 - {P}_{Pred} ) * {P}_{Cut \text{-} off} / ( 1 - {P}_{Cut \text{-} off} ) )$$

$${P}_{NCGS} = {P}_{Pred} / ( {P}_{Pred} + ( 1 - {P}_{Pred} ) * {Rp}_{Cut \text{-} off} )$$

$${P}_{NCGS} = {P}_{Pred} / ( {P}_{Pred} + {Rp}_{Cut \text{-} off} - {P}_{Pred} * {Rp}_{Cut \text{-} off} )$$

$${P}_{NCGS} = {P}_{Pred} / ( {P}_{Pred} ( 1 - {Rp}_{Cut \text{-} off} ) + {Rp}_{Cut \text{-} off} )$$

$${P}_{NCGS} = 1 / ( 1 - {Rp}_{Cut \text{-} off} + {Rp}_{Cut \text{-} off} / {P}_{Pred} )$$

$${P}_{NCGS} = 1 / ( 1 - {Rp}_{Cut \text{-} off} + {Rp}_{Cut \text{-} off} / 1 / ( 1 + {e}^{- ( score + constant )} ) )$$

$${P}_{NCGS} = 1 / ( 1 - {Rp}_{Cut \text{-} off} + {Rp}_{Cut \text{-} off} ( 1 + {e}^{- ( score + constant )} ) )$$

$${P}_{NCGS} = 1 / ( 1 - {Rp}_{Cut \text{-} off} + {Rp}_{Cut \text{-} off} + {Rp}_{Cut \text{-} off} {e}^{- ( score + constant )} )$$

$${P}_{NCGS} = 1 / ( 1 + {Rp}_{Cut \text{-} off} + {e}^{- ( score + constant )} )$$

$${P}_{NCGS} = 1 / ( 1 + {e}^{- ( score + constant )} {RP}_{Cut \text{-} off} )$$

$${P}_{IBSD} = 1 - {P}_{NCGS}$$

$${P}_{IBSD} = 1 - ( 1 / ( 1 + {Rp}_{Cut \text{-} off} {e}^{- ( score + constant )} ) )$$

$${P}_{IBSD} = ( 1 + {Rp}_{Cut \text{-} off} {e}^{- ( score + constant )} - 1 ) / ( 1 + {Rp}_{Cut \text{-} off} {e}^{- ( score + constant )} )$$

$${P}_{IBSD} = {Rp}_{Cut \text{-} off} {e}^{- ( score + constant )} / ( 1 + {Rp}_{Cut \text{-} off} {e}^{- ( score + constant )} )$$

$${P}_{IBSD} = 1 / ( 1 / ( {Rp}_{Cut \text{-} off} {e}^{- ( score + constant )} ) + 1 )$$

$${P}_{IBSD} = 1 / ( ( 1 / ( {Rp}_{Cut \text{-} off} ) {e}^{+ ( score + constant )} ) + 1 )$$

$${P}_{IBSD} = 1 / ( 1 + ( 1 / ( {Rp}_{Cut \text{-} off} ) {e}^{+ ( score + constant )} ) \\ {P}_{IBSD} = 1 / ( 1 + {e}^{- ( score + constant )} / {Rp}_{Cut \text{-} off} )$$

1. A method for diagnosing non celiac gluten sensitivity (NCGS) in a subject, comprising the following steps:
collecting clinical data indicating a degree of the severity (GS1, GS2) for one or more symptoms (S1, S2) perceived by said subject;
collecting biological data that are indicative of the zonulin concentration (ZL) in a serum sample of the subject being analysed;
elaborating said clinical and biological data to obtain a differentiation index (SC);
comparing said differentiation index with a threshold value (BC),
the NCGS being diagnosed when said differentiation index (SC) is greater than said threshold value (BC).

2. The method according to claim 1, wherein said one or more symptoms (S1, S2) include abdominal pain and abdominal distension.

3. The method according to claim 1, wherein the degree of severity (GS1, GS2) of said one or more symptoms in the range 0-4.

4. The method according to claim 1, wherein said clinical data are acquired through a questionnaire completed by said subject.

5. The method according to claim 1, wherein said amount of serum zonulin (LZ) is expressed as the ratio of the quantity by weight of said zonulin to the amount of total protein expressed in the serum of said subject.

6. The method according to claim 1, wherein said processing comprises a weighted sum of said clinical and biological data

7. The method according to claim 6, wherein said weighing sum is effected with a formula of the kind:
SC=C1*(LZ)+C2*(4−GS1)+C3*GS2
wherein:
SC represents the differentiation index;
LZ represents the amount of serum zonulin;
GS1 and GS2 represent the degree of severity of the symptoms considered; and
C1, C2, C3 are weight coefficients.

8. The method according to claim 7, wherein said weight coefficients (C1, C2, C3) are determined by means of a logistic regression analysis carried out on a reference population.

9. The method according to claim 7, wherein said coefficients are determined as:

| | |
|---|---|
| C₁ | 0.0457-0.0937 |
| C₂ | 0.3400-0.9208 |
| C₃ | 0.3576-0.9196 |

10. The method according to claim 9, wherein:
C₁=0.0697±0.0240
C₂=0.6304±0.2904, and
C₃=0.6386±0.2810.

11. The method according to claim 1, wherein said threshold value (BC) is determined as coinciding with the differentiation index corresponding to the maximum value of the curve of the likelihood ratio (LR) calculated for a reference population.

12. The method of claim 11, wherein said curvature of the likelihood ratio (LR) is obtained by interpolation.

13. The method according to claim 12, wherein said interpolation is carried out by means of a polynomial function, preferably of a third degree.

14. The method according to claim 1, wherein said threshold value (BC) is comprised between 2.802 and 3.984.

15. The method according to claim 14, wherein said threshold value (BC) is equal to 3.6760.

16. The method according to claim 1, further comprising a step for calculating a probability (P_{NCGS}) associated with the diagnosis of NCGS.

17. The method according to claim 1, wherein said probability (P_{NCGS}) associated with the diagnosis of NCGS is determined according to the formula:
P_{NCGS}=1/(1+e^{-(SC-2.6208)}*2.8724)
wherein 2.8724 represents an approximate value of a constant.

18. A computer program comprising a code for implementing, when running on a computer, a method according to claim 1.

19. A diagnostic kit for the diagnosis of non celiac gluten sensitivity (NCGS) in a subject, comprising:
a questionnaire for the acquisition of clinical data indicating a degree of the severity (GS1, GS2) for one or more symptoms (S1, S2) perceived by a subject; reagents for dosing the amount of serum zonulin (ZL) expressed in a serum sample of said subject.

20. A kit according to claim 19, further comprising a computer program for implementing, when running on a computer, a method for diagnosing NCGS in a subject, comprising the following steps:
collecting clinical data indicating a degree of the severity (GS1, GS2) for one or more symptoms (S1, S2) perceived by said subject;
collecting biological data that are indicative of the zonulin concentration (ZL) in a serum sample of the subject being analysed;
elaborating said clinical and biological data to obtain a differentiation index (SC);
comparing said differentiation index with a threshold value (BC), the NCGS being diagnosed when said differentiation index (SC) is greater than said threshold value (BC).
